(19) United States
(12) Patent Application Publication
(10) Pub. No.: US 2015/0320346 A1
(43) Pub. Date: Nov. 12, 2015

(54) Handheld Medical Instrument and System for Analyzing a Body Fluid

(71) Applicant: Roche Diabetes Care, Inc., Indianapolis, IN (US)

(72) Inventor: Ming-Wen Chen, Taipei City (TW)

(21) Appl. No.: 14/803,354

(22) Filed: Jul. 20, 2015

Related U.S. Application Data
(63) Continuation of application No. PCT/EP2014/052662, filed on Feb. 11, 2014.

(30) Foreign Application Priority Data
Feb. 11, 2013 (EP) 13154813.3

(51) Int. Cl. A61B 5/157; A61B 5/15

(52) U.S. Cl. CPC A61B 5/157; A61B 5/150022; A61B 5/150358

(57) ABSTRACT

The application concerns a handheld medical instrument for analyzing a body fluid, comprising a housing having a cassette compartment for receiving an exchangeable tape cassette, a transport mechanism comprising a manual drive which can be actuated by a user for rotating a tape spool of the tape cassette, such that a test tape of the tape cassette is wound forwards and functional elements stored thereon are provided for successive use, a stop device acting on the transport mechanism for a targeted stop of the test tape in a functional position of a functional element.

### CROSS REFERENCE TO RELATED APPLICATIONS

The present application is a continuation application of and claims priority to PCT Patent Application No. PCT/EP2014/052662, filed Feb. 11, 2014, which claims priority to European Patent Application No. 13154813.3, filed Feb. 11, 2013. The present application incorporates herein by reference the disclosure of each of the above-referenced applications in their entirety.

### TECHNICAL FIELD

Medical instruments, systems and methods for analyzing a body fluid are provided. In some implementations, the medical instrument is a handheld medical instrument that analyzes blood glucose tests.

### BACKGROUND

Diabetics are always attempting to tightly control their blood glucose levels so as to avoid the detrimental effects of their condition. High blood glucose levels, commonly referred to as hyperglycemia, can for example lead to organ damage, ketoacidosis, and/or long term debilitating or life-threatening conditions. If left untreated, low blood glucose conditions, commonly referred to as hypoglycemia, can lead to unconsciousness or even death. To avoid these problems, diabetics typically monitor their blood glucose levels closely and sometimes make adjustments to their treatment regimen so as to avoid hypoglycemia and hyperglycemia. Motorized devices are used in practice as blood glucose meters for the self-diagnosis of diabetics.

### SUMMARY

The present disclosure concerns a handheld medical instrument for analyzing a body fluid, such as for blood glucose tests. The medical instrument in at least one embodiment comprises a housing having a cassette compartment construed for receiving an exchangeable tape cassette and a transport mechanism comprising a hand-operated manual drive which can be operated by a user for rotating a tape spool of the tape cassette, such that a test tape of the tape cassette is wound forwards onto the tape spool and functional elements stored thereon on the test tape are provided for successive use. The present disclosure further concerns a system including an embodiment of the medical instrument and a disposable tape cassette, and a method of operation.

The reactive test fields, also referred to as functional fields, may be examined photometrically after the application of a small amount of blood sample in order to determine the glucose content as exactly and reliably as possible. Such tape cassettes are intended to be inserted as a disposable part into a compact hand-held device housing in order to allow the necessary analytical steps to be carried out automatically and rapidly. For a simplified system design, it has already been proposed in EP-A 1 702 565 to use a tape cassette which can be hand-operated by means of a lever arranged externally of a device housing.

### BRIEF DESCRIPTION OF THE DRAWINGS

The present disclosure is further elucidated in the following on the basis of an exemplary embodiment shown schematically in the drawings, where

FIG. 1 is a top view of a cassette-type blood glucose meter with test tape transport mechanism including a hand-operated drive;

FIG. 2 is a block diagram of the blood glucose meter;

FIG. 3 is a schematic view of a stop device acting on the tape transport mechanism;

FIG. 4 is a more detailed top view of the tape transport mechanism in cooperation with the stop device in an initial position;

FIG. 5 is an expanded view of FIG. 4;

FIGS. 6 to 8 show sequential positions following the initial position shown in FIG. 4; and

FIG. 9 is a top view of a section of the test tape.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that embodiments of the present disclosure are not limited to the precise arrangements shown.

### DETAILED DESCRIPTION

In at least one embodiment of the present disclosure, a medical instrument is provided. In at least one embodiment, the medical instrument comprises a housing having a cassette compartment configured to receive a tape cassette; a transport mechanism comprising a manual drive which can be actuated by a user in order to rotate a tape spool of the tape cassette, wherein a test tape of the tape cassette is wound forwards and functional elements stored thereon are provided for successive use; and a stop device acting on the transport mechanism and designed to stop the test tape in a functional position of a functional element.

Embodiments of the present disclosure are based on the idea of using an automatic device for securing proper tape positions. Accordingly, a stop device acting on the transport mechanism and designed to stop the test tape in a functional position of a functional element is described. The stop device is constructed as an instrument unit to interrupt the tape transport if needed. Thereby, the handling is kept simple for the user, who can concentrate his attention to the drive operation. Moreover, it is possible to assume exact functional positions thereby guaranteeing reliable measurements even under conditions of varying functional positions and tape transport distances during consecutive tests. Then, the user need not dispense with improved convenience provided by a large number of tests storable on a tape and easiness of disposal of used material on a waste tape spool. Manufacture of embodiments of the instrument may be simplified as compared to automatically driven devices, and the risk of a motor stall is avoided. Overall, the power consumption of the device can be minimized and high power internal supply is superfluous.

In at least one embodiment, the stop device has a position detecting unit preferably comprising a contact-free scanning sensor adapted for detecting a functional position of a functional element on the test tape. Thus, a targeted stop may be achieved even in case of tape slippage or distance tolerances of the functional elements on the tape. Non-contact scanning also allows for exact positioning without need for complicated tape design or mechanical engagement. The position detecting unit may directly or indirectly detect the functional element in a given position. In the latter case, the stop device may have an optical sensor adapted to detect position marks on the test tape and to provide a trigger signal to stop the test tape.

At least one embodiment of the present disclosure provides that the stop device has a locking unit which can be actuated for mechanical blocking of the transport mechanism. This provides a secure stop with simple measures.

In order to further allow a precise actuation, in at least one embodiment the stop device has a solenoid-operated locking actuator and a return spring to provide a locking force against rotation of the tape spool when the actuator is released.

The stop device in at least one embodiment has a control member which is linked to the manual drive to prevent a blocking of the transport mechanism in an initial transport phase. In such and embodiment, it is also possible to avoid excessive power consumption.

In order to prevent excessive forces on the test tape, the transport mechanism may in some embodiments be switched to an idle state when reaching a functional position by means of the stop device.

For a further reduction of the constructional effort it is also conceivable that the stop device has a position indication unit for providing a visual, acoustical or haptic perceptible instruction to the user to stop actuation of the manual drive, thus acting on the transport mechanism in a very simple way via the user.

A further improvement in safe and reliable hand-operation may be achieved when the manual drive comprises a swivel-mounted or linear guided push bar which can be operated by the user and a pinion which engages teeth on the push bar and converts the motion of the push bar into a rotational motion to rotate the tape spool.

A further improvement in this regard is based on a transport mechanism having a freewheel assembly that allows a rotary motion transferred to the tape spool in only one direction while preventing motion in the opposite direction.

For a reliable torque transmission, at least one embodiment of the transport mechanism has a gear-wheel which can be rotated by the manual drive and which can be blocked by the stop device, and wherein the gear-wheel is directly coupled to the tape spool by a connecting journal when the tape cassette is inserted in the cassette compartment.

In order to avoid excessive loads, at least one embodiment of the transport mechanism has a speed indication unit for providing a visual, acoustical or haptic perceptible signal to the user to indicate the rotational speed of the tape spool.

In at least one embodiment of the present disclosure, the transport mechanism includes an electric energy generator which can be operated by the manual drive and an electric motor supplied with energy from the generator preferably via a super capacitor as a storage means.

For further automation of the positioning, at least one embodiment of the transport mechanism includes a storage means for storing mechanical energy, preferably a spring which can be loaded by the manual drive in order to automatically rotate the tape spool in a transport cycle.

In order to secure reliable measurements, at least one embodiment of the test tape may have consecutive tape sections each including a functional element formed as a test field for application of body fluid and at least one additional functional element, specifically a calibration field. Generally, the term “functional element” as used herein denotes a distinctive element or field on the test tape which can be positioned by tape transport to assume a functional position in which an instrument operation, specifically a measurement mode is supported or triggered.

The present disclosure also concerns a medical system for analyzing a body fluid, in particular for blood glucose tests, comprising a tape cassette including a test tape and a handheld instrument, the instrument comprising:

a housing having a cassette compartment construed for receiving the tape cassette,

a transport mechanism comprising a manual drive which can be actuated by a user in order to rotate a tape spool of the tape cassette, such that the test tape is wound forwards and functional elements stored thereon are provided for successive use,

a stop device acting on the transport mechanism and designed to stop the test tape in a functional position of a functional element provided on the test tape.

In at least one embodiment of the present disclosure, a method for operating a handheld medical instrument for analyzing a body fluid is disclosed. The method, in at least one embodiment, comprises
inserting an exchangeable tape cassette comprising a test tape into a cassette compartment provided in a housing of the instrument,
actuating a manual drive of a transport mechanism in order to rotate a tape spool of the tape cassette, such that the test tape is wound forwards and functional elements stored thereon are provided for successive use,
stopping the test tape in a functional position of a functional element by means of an automatic stop device acting on the transport mechanism.

The drawings show at least one embodiment of a medical instrument configured as a portable blood glucose meter 10 for self-monitoring of blood glucose and comprising a housing 12 with a cassette compartment 14 defining a space for inserting a disposable tape cassette 16 and a transport mechanism 18 for rotating a tape spool 20 of the tape cassette 16, such that an analytical test tape 22 of the cassette can be wound forwards and functional elements 24 stored thereon can be sequentially provided for use, specifically for application and investigation of a blood sample.

As illustrated in FIG. 1, the transport mechanism 18 comprises a manual drive 26 which can be operated by a user in bidirectional movement as depicted by double arrow 28. The system including the tape cassette 16 may also be equipped with a lancing aid 30, allowing the user to draw a fresh sample of whole blood on-the-spot and to apply the sample at an application tip of the cassette 16. The on-board photometric detection of the analyte (glucose) is known per se in the state of the art and needs not be explained in further detail. The test result may be rapidly provided on a display 32. The tape spool 20 is intended for taking or winding-up the used section of the test tape 22, whereas a storage 32 shields unused tape preferably on an unwinding spool. Thus, the flexible test tape 22 is only transported via rotation of the spool 20, so that sprocket holes can be avoided in the thin tape foil.

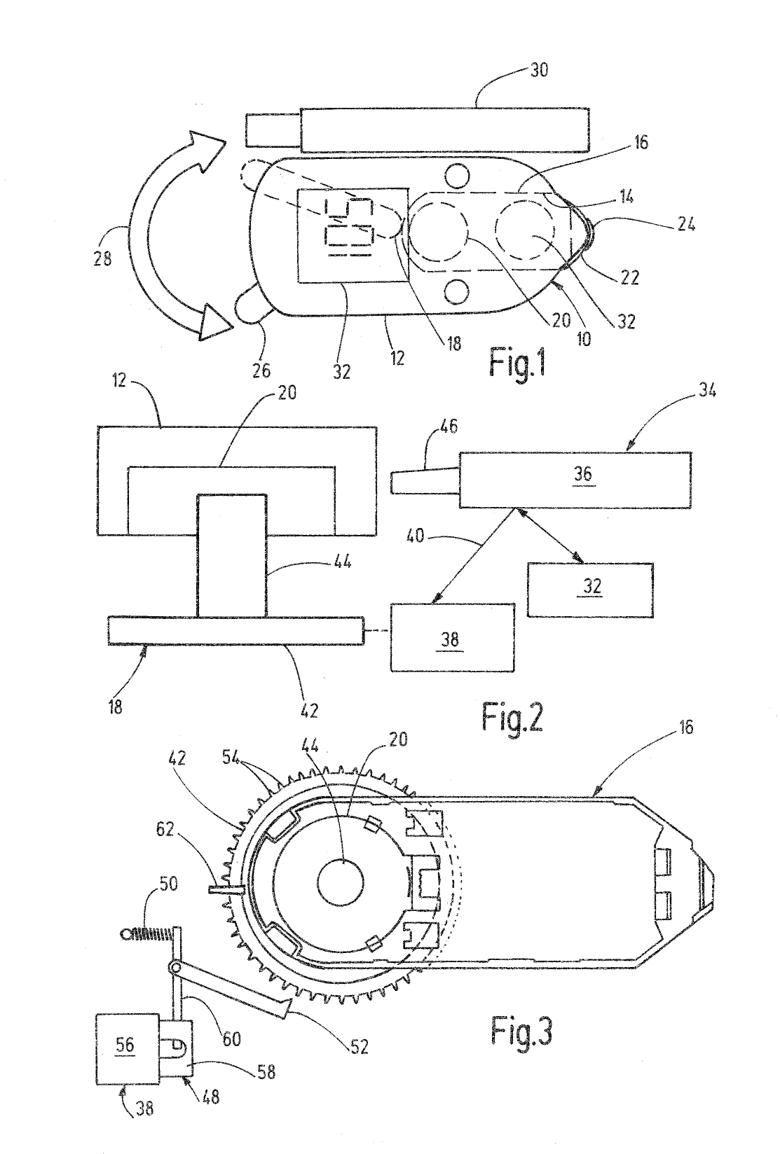

FIG. 2 further exemplifies that the instrument 10 comprises a stop device 34 that acts on the transport mechanism 18 to achieve a targeted stop of the test tape 22 in a functional position, for example when a functional element 24 in the form of a reactive test field is positioned on the tip of the cassette 16. The stop device 34 comprises a position detecting unit 36 scanning the test tape 22 during its transport and a locking unit 38 which can be actuated by a trigger signal 40 of the position detecting unit 36 for mechanical blocking of the transport mechanism 18. For this purpose, the transport mechanism 18 comprises a rotating unit 42, e.g. a gear-wheel which is directly coupled to the tape spool 20 by a connecting journal 44 when the tape cassette 20 is inserted in the housing 12.

In a practical embodiment, the position detecting unit 36 has an optical sensor 46 to detect position marks on the test tape 22, as explained in more detail further below. Furthermore, the position detecting unit 36 may be connected to the display 32 to give a positional feedback to the user. In a very simple arrangement, such feedback may be intended to cause the user to stop operating the manual drive 26, thereby acting on the transport mechanism via 18 the user.

FIG. 3 illustrates the function of the locking unit 38 of the stop device 34. The gear-wheel 42 directly drives the tape spool 20 of the tape cassette 16. The locking unit 38 comprises a solenoid-operated actuator 48 and a return spring 50 to provide a locking force via a latch 52 that engages the teeth 54 of the gear-wheel 42. In the unlocked state, the solenoid coil 56 is energized and the solenoid core 58 is retrieved (to the left in FIG. 3). At the same time, the return spring 50 is biased or extended, and the latch 52 is out of engagement, thereby allowing the gear-wheel 42 to rotate. Upon receiving a trigger signal 40, the coil 56 is de-energized and the core 58 is drawn out (to the right in FIG. 3) under the return force of the contracting return spring 50. As the lever 60 linking the actuator 48 and the spring 50 is tilted, the latch 52 locks against the teeth 52 and prevents further motion of the gear-wheel 42.

It may be desirable to let the user feel the proper rotational speed when operating the manual drive. In a very simple embodiment, gear-wheel 42 as shown in FIG. 3 may form a dial for a direct manual rotation. Then, a mark spring 62 on the gear-wheel 42 can provide a haptic perceptible signal or feedback to the user to indicate the present revolutions or speed of rotation.

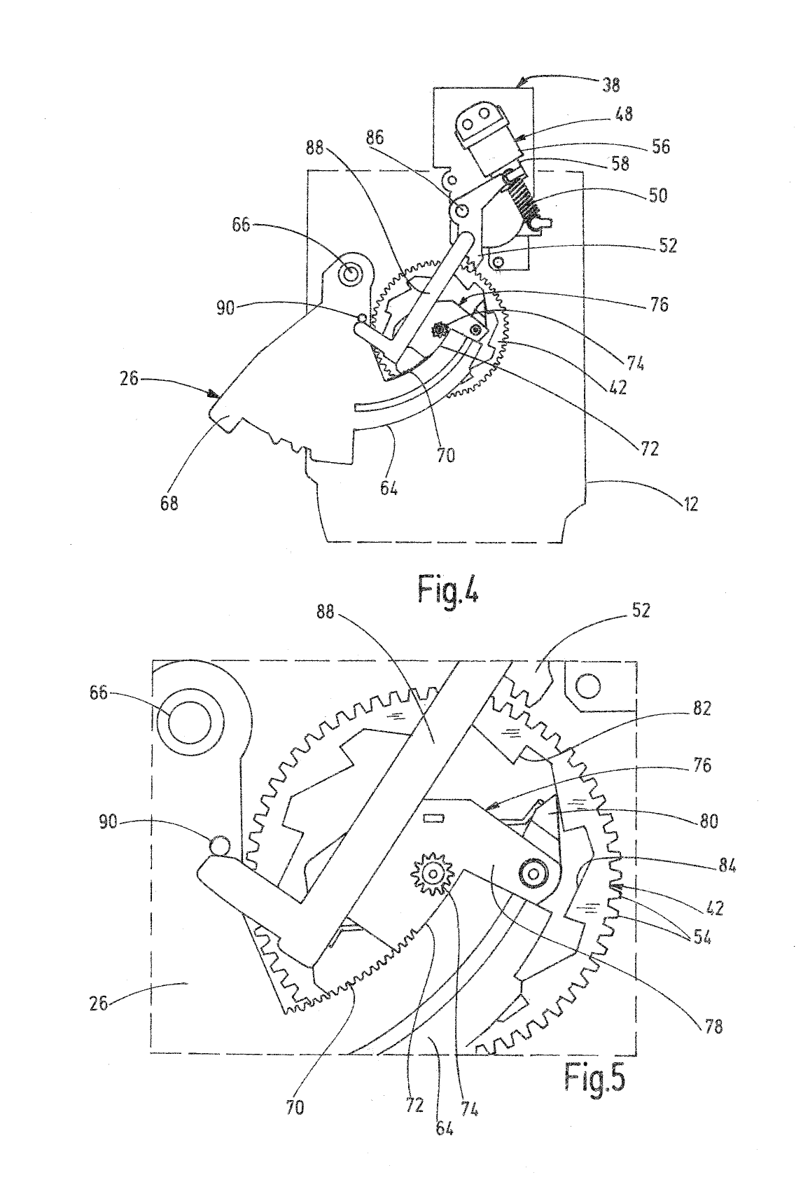

FIG. 4 shows a manual drive 26 comprising a push-bar 64 which is swivel-mounted at a swivel-bearing 66 and can be hand-operated by means of a handle 68. The push-bar 64 has an arched toothed segment 70 and an override segment 72 without teeth towards its free end.

As can be also seen from FIG. 5, a rotatable pinion 74 is mounted in the movement path of the push-bar 64, so that it can be rotated by the toothed segment 70. The pinion 74 is connected to a freewheel assembly 76 in a rotatably fixed manner. The freewheel assembly 76 has a polygonal freewheel body 78 which is provided at opposed ends with two swivel-mounted pawls 80. The pawls 80 are springloaded against the inner asymmetrically saw-toothed contour of the gear-wheel 42, which is connectable to the tape spool 20 as explained above.

In the course of manual operation, when the handle 68 is pushed inwards to the housing 12, the pinion 74 eventually engages the toothed segment 70 and converts the motion of the push-bar 64 into a rotation of the body 78 in anticlockwise direction. The pawls 80 will then catch against the first steeply sloped inner edge 80 of the gear-wheel 42, thereby locking it for cooperative movement with the freewheel body 78. When the push-bar 64 is pivoted in the opposite (outward) direction, however, the freewheel body 78 rotates in clockwise direction and the pawls 80 can easily slide up and over the gently sloped chamfers 84 of the gear-wheel 42, thereby allowing a free rotation of the assembly 76 without actuation of the gear-wheel 42.

Turning back now to FIG. 4 the locking unit 38 of the stop device 34 is shown to have a similar arrangement as already explained above. The solenoid-operated actuator 48 is connected at its core 58 to return spring 50, which is expanded in the initial state. The latch 52 is formed as a two-armed lever which is swivel-mounted at swivel-bearing 86. In the initial state, the latch 52 does not engage the teeth 54 of the gear-wheel 42.

In order to secure this initial state independently of the current feed state of the solenoid coil 56, the latch 52 is linked to the push-bar 68 of the manual drive 26 by means of an L-shaped control arm 88. This arm 88 is supported at its shorter bracket by means of a support pin 90 projecting at the side of the push-bar 64. Thereby, the return force of the return spring 50 can be held solely mechanical, without current consumption in the coil 56.

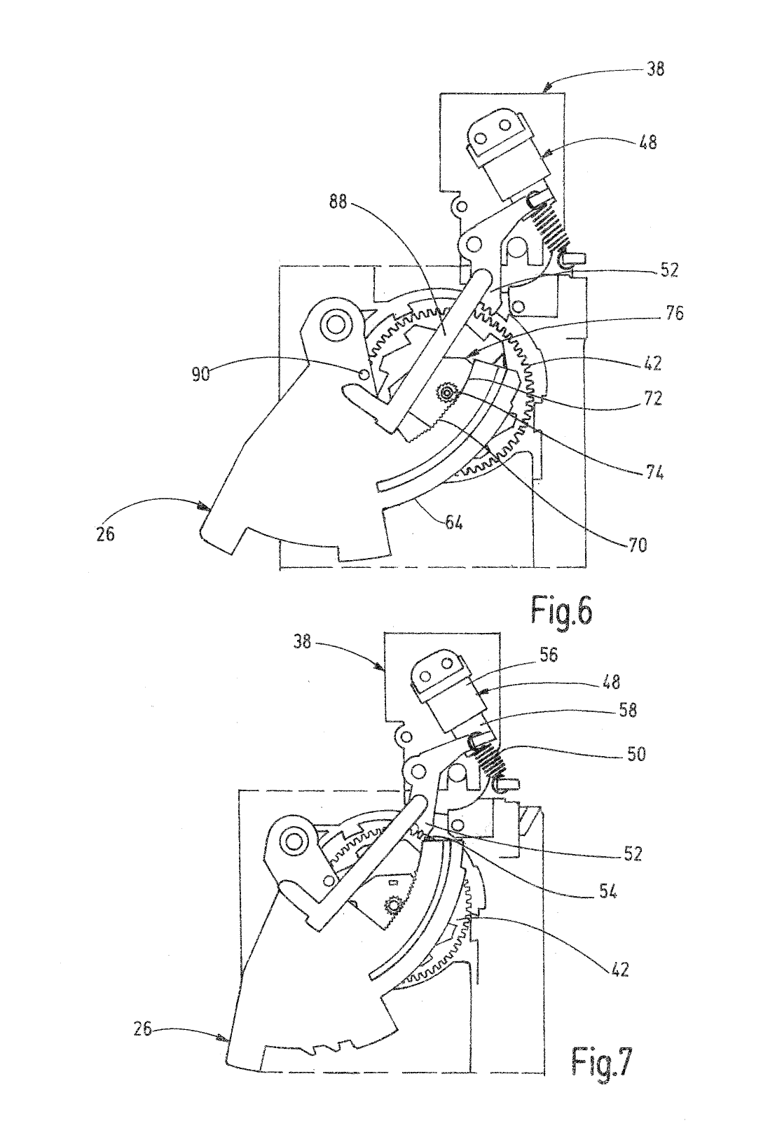

As apparent from FIG. 6, in the beginning phase of the manual operation of the push-bar 64, the tooth-free override segment 72 moves past the pinion 74 without engagement, however, allowing the control arm 88 to come clear from the support pin 90. In this phase, the solenoid-operated actuator 48 should be already energized to prevent unwanted blocking of the gear-wheel 42. The current feed may be achieved by means of a switch (not shown) actuated by the push-bar 64. Upon further movement of the push-bar 64, the gear-wheel 42 is rotated through the toothed segment 70, and the test tape 22 is advanced by means of the tape spool 20.

FIG. 7 shows the blocking state in which the locking unit 38 has received a trigger signal, and the solenoid-operated actuator 48 is switched-off. Then, the return spring 50 contracts, whereby the core 58 is drawn out of the coil 56, and the latch 52 is pivoted into the blocking state where the teeth 54 of the gear-wheel 42 are engaged. Thereby, the test tape 22 is stopped in a functional position.

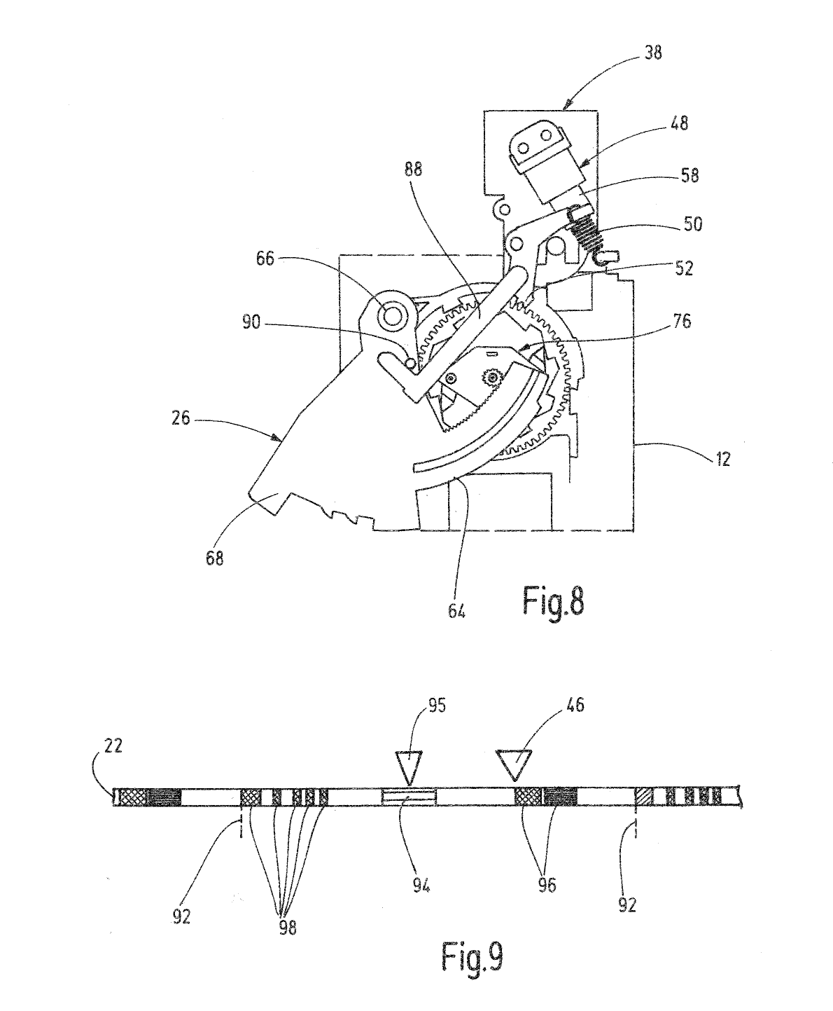

FIG. 8 shows a return state in which the push-bar 64 has swung back (outwards of the housing 12) under the influence of a torsion spring which may be arranged in the bearing 66. This motion occurs automatically when the user releases the handle 68, however only to an angle distance where the support pin 90 catches the control arm 90 in the center of the short bracket. Then, in order to reach the initial state shown in FIG. 4, the user has to actively swivel out the handle 68, whereby the return spring 50 is extended, the core 58 is moved back and the latch 52 is secured through the control arm 88 supported at pin 90.

FIG. 9 illustrates an exemplary test tape 22 that has consecutive sections 92 (for example, a total of fifty sections 92) that are each used for a single measurement. Body fluid is supplied to an analytical test field 94 and investigated by means of an optical measurement unit 95 of the instrument 10 in a defined functional (measurement) position. In addition, calibration fields 96 are provided in each section 92 which can be used for a calibration of the measurement unit 95 when properly aligned. For a targeted stop of the test tape 22, each section 92 contains position marks 98 which can be detected by the optical sensor 46 of the position detection unit 36. Apparently, the tape transport distances vary in order to approach the different functional positions of the functional elements 24, i.e. the test field 94 and the calibration fields 96. Moreover the diameter of the tape wrap on the tape spool 20 increases with increasing use of tests. These difficulties can be overcome by the cooperative action of the stop device 34 and the manual drive 26.

While various embodiments of medical instruments, systems and methods for analysing a body fluid have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. Handheld medical instrument for analyzing a body fluid, the handheld medical instrument comprising:
a housing having a cassette compartment configured to receive a tape cassette;
a transport mechanism comprising a manual drive which can be actuated by a user in order to rotate a tape spool of the tape cassette, wherein a test tape of the tape cassette is wound forwards and functional elements stored thereon are provided for successive use; and
a stop device acting on the transport mechanism and designed to stop the test tape in a functional position of a functional element.

2. The handheld medical instrument of claim 1, wherein the stop device has a position detecting unit.

3. The handheld medical instrument of claim 2, wherein the position detecting unit comprises a contact-free scanning sensor adapted for detecting a functional position of a functional element provided on the test tape.

4. The handheld medical instrument of claim 1, wherein the stop device has an optical sensor adapted to detect position marks on the test tape and to provide a trigger signal to stop the test tape.

5. The handheld medical instrument of claim 1, wherein the stop device has a locking unit which can be actuated for mechanical blocking of the transport mechanism.

6. The handheld medical instrument of claim 1, wherein the stop device has a solenoid-operated locking actuator and a return spring to provide a locking force against rotation of the tape spool when the locking actuator is released.

7. The handheld medical instrument of claim 1, wherein the stop device has a control arm which is linked to the manual drive to prevent a blocking of the transport mechanism in an initial transport phase.

8. The handheld medical instrument of claim 1, wherein the transport mechanism can be switched to an idle state when reaching a functional position by means of the stop device.

9. The handheld medical instrument of claim 1, wherein the stop device has a position indication unit for providing a visual, acoustical or haptic perceptible instruction to the user to stop actuation of the manual drive.

10. The handheld medical instrument of claim 1, wherein the manual drive comprises a swivel-mounted or linear guided push bar which can be operated by the user and a pinion which engages teeth on the push bar and converts the motion of the push bar into a rotational motion to rotate the tape spool.

11. The handheld medical instrument of claim 1, wherein the transport mechanism has a freewheel assembly that allows a rotary motion transferred to the tape spool in only one direction while preventing motion in the opposite direction.

12. The handheld medical instrument of claim 1, wherein the transport mechanism has a gear-wheel which can be rotated by the manual drive and which can be blocked by the stop device, and wherein the gear-wheel is directly coupled to the tape spool by a connecting journal when the tape cassette is inserted in the cassette compartment.

13. The handheld medical instrument of claim 1, wherein the transport mechanism has a speed indication unit for providing a visual, acoustical or haptic perceptible signal to the user to indicate the rotational speed of the tape spool.

14. The handheld medical instrument of claim 1, wherein the transport mechanism includes an electric energy generator which can be operated by the manual drive and an electric motor supplied with energy from the generator preferably via a super capacitor as a storage means.

15. The handheld medical instrument of claim 1, wherein the transport mechanism includes a storage means for storing mechanical energy, preferably a spring which can be loaded by the manual drive in order to automatically rotate the tape spool in a transport cycle.

16. The handheld medical instrument of claim 1, wherein the test tape has consecutive tape sections each including a functional element formed as a test field for application of body fluid and at least one additional functional element, specifically a calibration field, and wherein different tape transport distances are required to reach a respective functional position of the functional elements in each tape section.

17. A medical system for analyzing a body fluid, in particular for blood glucose tests, comprising a tape cassette including a test tape and a handheld instrument, the handheld instrument comprising:
a housing having a cassette compartment configured to receive the tape cassette;
a transport mechanism comprising a manual drive which can be actuated by a user in order to rotate a tape spool of the tape cassette, such that the test tape is wound forwards and functional elements stored thereon are provided for successive use; and
a stop device acting on the transport mechanism and designed to stop the test tape in a functional position of a functional element provided on the test tape.

18. A method for operating a handheld medical instrument for analyzing a body fluid, the method:
inserting an exchangeable tape cassette comprising a test tape into a cassette compartment provided in a housing of the handheld medical instrument, the handheld medical instrument further comprising a transport mechanism having a manual drive;
actuating the manual drive of the transport mechanism in order to rotate a tape spool of the tape cassette, such that the test tape is wound forwards and functional elements stored thereon are provided for successive use; and
stopping the test tape in a functional position of a functional element by means of an automatic stop device acting on the transport mechanism.
